(19) United States
(12) Patent Application Publication
(10) Pub. No.: US 2020/0160961 A1
(43) Pub. Date: May 21, 2020

(54) METHOD FOR RESPONDING TO EARLY ABANDONMENT OF AN EXERCISE SESSION BY A USER

(71) Applicant: Aaptiv Inc., New York, NY (US)

(72) Inventors: Amit Wadhawan, New York, NY (US); Max Simon, New York, NY (US); David Cohen, New York, NY (US); Chris Fischer, New York, NY (US); Ethan Agarwal, New York, NY (US); Kathleen Yanolatos, New York, NY (US)

(21) Appl. No.: 16/678,972

(22) Filed: Nov. 8, 2019

Related U.S. Application Data
(60) Provisional application No. 62/757,542, filed on Nov. 8, 2018.

(51) Int. Cl. G16H 20/30; A63B 71/06; A63B 24/00

(52) U.S. Cl. CPC G16H 20/30; A63B 71/0622; A63B 24/0075; A63B 2220/62; A63B 2071/063; A63B 2071/0694; A63B 2024/0068; A63B 24/0059

(57) ABSTRACT

One variation of a method includes: serving a class—spanning a duration of time, including an audio track of a trainer narrating an exercise routine of a first type, and associated with a first difficulty level—to a user; if the user exits the class within a first time window during replay of the class, presenting a first list of classes—spanning durations approximating the duration and including audio tracks of trainers narrating exercise routines of the first type—to the user; and, if the user exits the class within a second time window succeeding the first time window, serving a prompt to select a second class—from a second list of classes—spanning durations less than the first duration and including audio tracks of trainers narrating exercise routines of difficulty levels less than the first difficulty level—for completion during a remainder of the exercise session to the user.

### CROSS-REFERENCE TO RELATED APPLICATIONS

This Application claims the benefit of U. S. Provisional Application No. 62/757,542, filed on 8 Nov. 2018, which is incorporated in its entirety by this reference.

### TECHNICAL FIELD

This invention relates generally to the field of fitness and more specifically to a new and useful method for responding to early abandonment of an exercise session by a user in the field of fitness.

### BRIEF DESCRIPTION OF THE FIGURES

FIG. 1 is a flowchart representation of a method;

FIG. 2 is a graphical representation of one variation of the method; and

FIG. 3 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

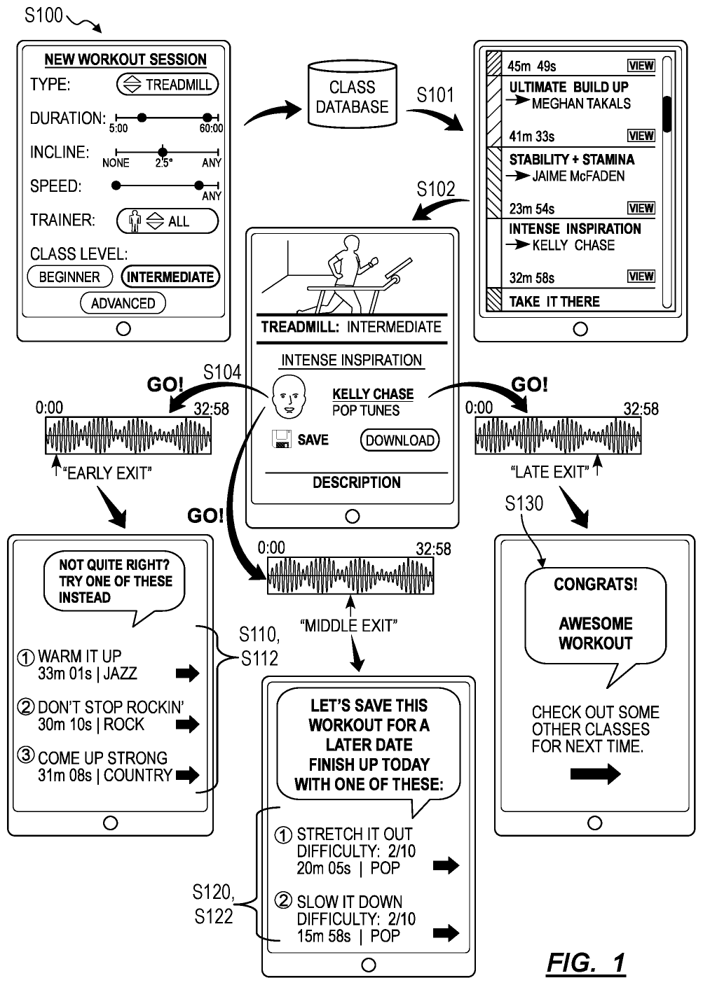

As shown in FIG. 1, a method S100 for responding to early abandonment of exercise by a user includes: at a computing device accessed by the user at a start of an exercise session, receiving selection of a first class from a corpus of prerecorded classes, the first class spanning a first duration of time, comprising a first audio track of a first trainer narrating a first exercise routine of a first type, and associated with a first difficulty level in Block S102; and serving the first class to the user through the computing device in Block S104. The method S100 also includes, in response to the user exiting the first class at the computing device within a first time window during replay of the first class: aggregating a first list of classes, from the corpus of prerecorded classes, comprising audio tracks of trainers narrating exercise routines of the first type in Block Silo; and presenting the first list of classes to the user through the computing device in Block S112. The method S100 further includes, in response to the user exiting the first class at the computing device within a third time window succeeding the first time window during replay of the first class: serving a congratulatory prompt, for completing the first exercise routine, to the user through the computing device in Block S130. The method S100 also includes, in response to the user exiting the first class at the computing device within a second time window between the first time window and the third time window during replay of the first class: aggregating a second list of classes, from the corpus of prerecorded classes, comprising audio tracks of trainers narrating exercise routines of difficulty levels less than the first difficulty level and spanning durations less than the first duration of time in Block S120; and serving a prompt to select a second class, from the second list of classes, for completion during a remainder of the exercise session to the user through the computing device in Block S122.

One variation of the method S100 includes: at a first computing device accessed by a first user at a start of a first exercise session, receiving selection of a first class from a corpus of prerecorded classes, the first class spanning a first duration of time, comprising a first audio track of a first trainer narrating a first exercise routine of a first type, and associated with a first difficulty level in Block S102; and serving the first class to the first user through the first computing device in Block S104. This variation of the method S100 also includes, in response to the first user exiting the first class at the first computing device within a first time window during replay of the first class: aggregating a first list of classes, from the corpus of prerecorded classes, comprising audio tracks of trainers narrating exercise routines of the first type in Block Silo; and presenting the first list of classes to the first user through the first computing device in Block S112. This variation of the method S100 further includes, at a third computing device accessed by a third user at a start of a third exercise session, receiving selection of the first class in Block S102; serving the first class to the third user through the third computing device in Block S104; and, in response to the third user exiting the first class at the third computing device within a third time window succeeding the first time window during replay of the first class, serving a congratulatory prompt, for completing the first exercise routine, to the third user through the third computing device in Block S130. This variation of the method S100 also includes, at a second computing device accessed by a second user at a start of a second exercise session, receiving selection of the first class in Block S102; and serving the first class to the second user through the second computing device in Block S104. This variation of the method S100 further includes, in response to the second user exiting the first class at the second computing device within a second time window between the first time window and the third time window during replay of the first class: aggregating a second list of classes, from the corpus of prerecorded classes, comprising audio tracks of trainers narrating exercise routines of difficulty levels less than the first difficulty level and spanning durations less than the first duration of time in Block S120; and serving a prompt to select a second class, from the second list of classes, for completion during a remainder of the second exercise session to the second user through the second computing device in Block S122.

### 2. Applications

Generally, the method S100 can be executed by a computer system: to serve a pre-recorded audio fitness class to a user through a computing device during a exercise session; to track the user's progress through this first class during the exercise session; and to intelligently respond to the user's exit from the class in (near) real-time in order to guide the user toward selecting a more interesting alternate class, support the user toward engaging with a more appropriate class rather than abandoning the exercise session as a whole, or congratulate and prepare the user for a next exercise session based on when and how the user exits the first class.

In particular, the computer system can include a remote server that hosts or interfaces with a class database containing a corpus of prerecorded classes, each containing an audio track of a trainer narrating an exercise routine (e.g., orally communicating instructions for the exercise routine) and including music (or another audio track) throughout its duration. The computer system can also host or interface with a native application or web browser executing on a user's computing device (e.g., a smartphone, a tablet, a smart television): to receive selections from the class database; to serve selected classes to the user; and to selectively “catch” a user exiting from a selected class by serving a short, filtered list of alternate classes that may be more relevant to the user and/or to congratulate the user for her performance according to the method S100 and based on data collected at the computing device. The computer system can therefore execute the method S100 remotely and interface with the user through the native application or web browser. (Additionally or alternatively, the native application or web browser can execute Blocks of the method S100 locally at the computing device.)

### 3. Applications: Example

In one example, the computer system: ranks classes—in a corpus of classes on the fitness platform—by probability that the user will complete these classes based on historical class completion data of the user (e.g., class types, difficulty levels, music genres, trainer styles, and/or durations of classes previously completed by the user) and/or based on historical class completion data of other similar users on the fitness platform; generates an initial list of a subset of these classes associated with greatest ranks; and queues this initial list of classes for presentation to the user.

Upon entering a gymnasium, a fitness center, a professional exercise studio, a home or office exercise studio, etc. or stepping onto a field or track at the start of an exercise session, a user may: open the native application (or web browser) executing on a computing device (e.g., a smartphone, tablet, or other handheld or wearable device); review this initial list of classes within the native application; and then select—from this initial list—a first class for this exercise session. In this example, the user may also enter filter parameters, such as class type (e.g., running, weightlifting, yoga, cycling or spinning), difficulty level, type of instruction (e.g., fun, focused), duration, music genre, trainer style, trainer demographic, etc. The native application (or the computer system) can then: filter the corpus of prerecorded classes; generate a revised list of classes that meet these filter parameters and that are associated with the greatest ranks; and present this revised list of classes to the user. The user may also review titles and textual descriptions of these classes before selecting a first class for this exercise session.

Upon selection of the first class by the user at the native application, the native application can retrieve (e.g., download, stream) this first class from the class database and initiate playback of this first class—such as an audio track and/or a video track—as shown in FIG. 1. Throughout this exercise session, the user may follow along to trainer instructions and trainer guidance thus presented in this first class.

However, at some time prior to completion of the first class, the user may determine that she is not satisfied with the first class, is not or is no longer interested in the first class, or is frustrated with the first class. Accordingly, the user may elect to exit (or “abandon”) the first class. By exiting the first class before its completion, the user may receive minimal short-term benefit from any physical activity described or presented in the first class. Furthermore, failure to maintain motivation to complete the first class may frustrate the user and reduce the user's likelihood of returning to the native application (or web browser), selecting another class from the database, and completing a next class in the future, which may negatively impact the user's ability to achieve her longer-term fitness goals.

However, the user may have already dedicated time (e.g., 30-40 minutes) to this exercise session, and some or much of this time may remain available to the user for further exercise even after the user exits the first class. Therefore, the computer system (and/or the native application) can execute Blocks of the method S100: to automatically identify a small set of classes (e.g., one class, three classes) to fill the user's remaining time and that are predicted to better match the user's needs, interests, and/or expectations at the current time; present this small set of classes to the user in (near) real-time following the user's exit from the first class; and to prompt the user to select an alternate class—from this small set of classes—to complete during the remainder of this exercise session. The computer system can thus support the user toward completing a class—of the same or dissimilar type and of similar or (significantly) reduced difficulty—during this exercise session, which may: improve the user's confidence; improve the user's motivation to complete a short-term exercise goal (e.g., exercise during the current exercise session); and support the user toward a longer-term fitness goal.

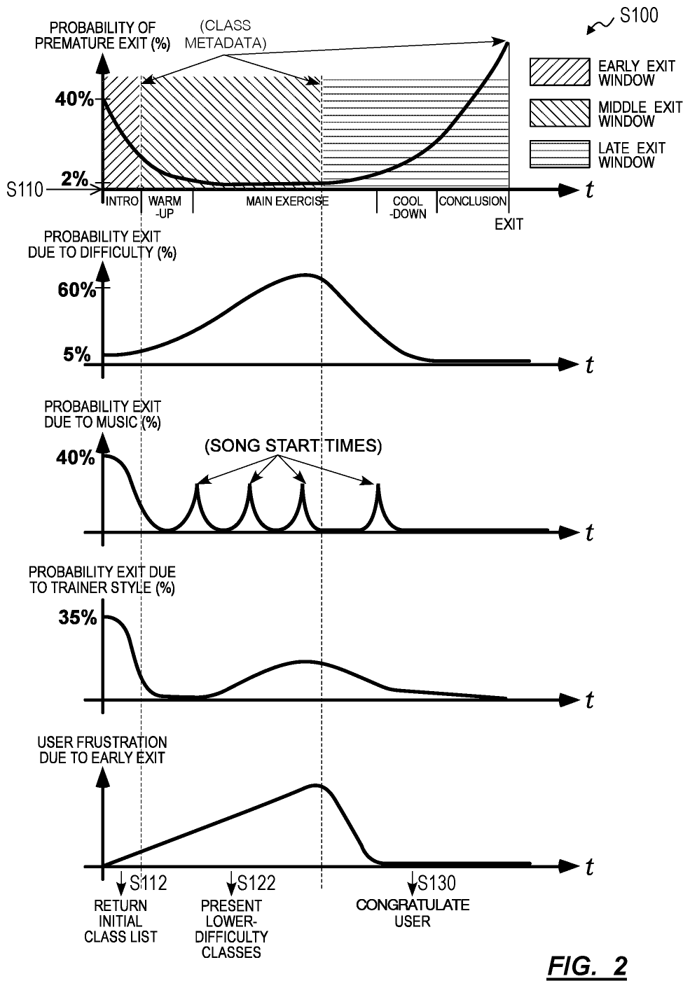

Furthermore, the computer system can execute Blocks of the method S100 to predict a reason for the user's early exit from the first class—such as based on a proportion of the first class completed by the user at time of exit, as shown in FIG. 2—and then serve targeted guidance to the user and/or aggregate a subset of classes to recommend to the user responsive to this reason. For example, a first user who exits the first class within the first 30 seconds of the first class (or within the first 2% of the total duration of the first class) may have quickly screened (or “surveyed”) the first class, determined that she is not particularly interested in this first class, and exited the first class with the intention of selecting an alternate class and with limited or no sense of frustration or reduced motivation to complete a class during the current exercise session. Such action by the first user may thus represent a “low-risk” “early exit” from the first class. The computer system can therefore compile and serve a short list of alternate classes spanning similar durations, of the same class type, and/or characterized by difficulties similar to those of the first class that the first user recently exited. However, because the first user may have exited the first class due to dislike of a music genre, trainer, or trainer style, the computer system can populate this list of alternate classes with classes containing music of different genres and/or associated with a different trainer than the first class.

In contrast, a second user who exits the first class at a time between 8% and 50% of the duration of the first class may have done so as a result of frustration with the first class, such as resulting from excessive difficulty for the second user, disagreement with a comment from the trainer narrating the first class, dislike of a song or lyric played back during the first class, or need for a different level of support or positive reinforcement from the trainer. The second user may also feel more commitment and interest in completing the first class given this greater duration of time that the she remained in the first class. Therefore, the second user's later exit from the first class may result in a greater sense of frustration for the second user, such as frustration from her failure to complete the exercise and/or frustration resulting from a different between the second user's expectations for the class and the experience delivered by the first class. The second user's frustration from exiting the first class may therefore pose risk to achieving both a short-term exercise goal (e.g., completing an exercise during the current exercise session) and a longer-term fitness goal (e.g., building a positive habit and consistency around fitness) for the second user. This action by the second user may thus represent a “high-risk” “middle exit.” The computer system can therefore: compile a list of alternate classes spanning the remaining duration of the exited class, of the same type (e.g., running, weight lifting, or spinning) or of a “cool-down” type (e.g., stretching, yoga, meditation), characterized by difficulties less than that of the first class the second user recently exited, and/or associated with a different (e.g., more supportive) trainer, etc.; and serve this list of alternate classes to the second user via the native application in (near) real-time, thereby immediately presenting the second user with a viable option for recovering from this middle exit. In particular, the computer system can: leverage the second user's interactions with the first class and characteristics of the first class to automatically filter a large database (e.g., thousands) of classes down to a small number of (e.g., three) classes predicted to be better suited for the second user's current needs, interests, and fitness level; and present this small number of classes to the second user with a prompt to continue her workout with an alternative class from this list. The computer system (or the native application) may thus reduce the second user's frustration, maintain the second user's engagement with the native application, and increase the second user's likelihood of completing an exercise during this exercise session, completing future exercise sessions, and achieving her fitness goals.

Furthermore, a third user who exits the first class at a later time (e.g., after completing 50% of the total duration of the first class) may have done so as a result of completing a main exercise period of the first class and lacking interest in a cool-down period, stretching period, or exercise summary from the trainer upon conclusion of the primary exercise in this first class. This decision by the third user to leave the first class before its conclusion may thus represent a “low-risk” “late exit.” The computer system can therefore serve—to the third user—a notification congratulating the third user for her effort and prompting the third user to complete a survey for the first class (e.g., by reporting perceived difficulty, interest, and satisfaction with the first class) through the native application. The computer system can also leverage the third user's responses to this survey to compile a list of classes that may be particularly relevant to the third user during a next exercise session and then prompt the third user to select a class—from this set of classes—for a next exercise session, such as: before the user's closes the native application; and/or when the user opens the native application at the start of a next exercise session at a later date.

The computer system (and/or the native application) can therefore execute Blocks of the method S100 to immediately respond to abandonment of a first class by a user, including selecting and recommending another class that the user may be more likely to complete during the remainder of the current exercise session or during a next exercise session based on characteristics of premature exit from the first class by the user. In particular, the computer system (and/or the native application) can execute Blocks of the method S100 to leverage implicit feedback from the user in the form of premature class abandonment: to identify a subset of classes predicted to limit or reduce the user's frustration; to present these classes to the user when the user is at greatest risk of disengagement from the fitness platform and from her fitness goals; and to thus support the user toward building positive habits (e.g., consistency) around exercise and fitness.

### 4. Classes

As described above, the method S100 can be executed by a remote computer system and/or a native application in conjunction with a class database (hereinafter, collectively, the “system”) to form a fitness platform accessible by users to view and follow along to instructional exercise and fitness sessions. The class database can store a corpus of pre-recorded classes that can be selected by users and downloaded to computing devices (e.g., smartphones, tablets, smart televisions) associated with these users.

In one implementation, a class includes an audio track containing guided oral instruction from a trainer narrating an exercise routine, music, and metadata describing characteristics of the exercise routine, the trainer, and/or the music, etc. For example, a class can be created by recording an audio track of a trainer narrating and providing oral instruction for narrating an exercise routine. During post-production of the audio track, the audio track can be augmented with music (e.g., a series of song titles); and a title and a brief description of the class can be stored in metadata of this audio track.

Additional data for each song title in the post-production audio track can be written to metadata of the audio track, such as: a start time of the song title (e.g., in the form of timestamps from the start of the audio track); the artist, genre or style, and duration of the song title; whether and when the song title includes profanity (e.g., in the form of timestamps from the start of the song title); pitch (e.g., melody, harmony) of the song title; rhythm (e.g., tempo, meter, articulation) of the song title; dynamics (e.g., loudness and softness) of the song title; sonic qualities (e.g., timbre, texture, color) of the song title; and/or components (e.g., instrumental accompaniment, vocal accompaniment) of the song title; etc.

Similarly, trainer-related data can be written to the class metadata, such as including: the age; gender; other demographic; voice characteristics (e.g., vocal timbre, vocal registration); instruction style (e.g., tailored for beginners or advanced users); and trainer mood or personality (e.g., motivating or “drill sergeant,” calming or aggressive, focused or fun); etc.

Furthermore, a class may be structured to include: an intro in which a trainer explains the nature of the class, such as including a difficulty level, a duration, and types of exercises presented in the class; a warm up period including stretching and/or light motion; a main exercise period representing a core component of the class; a cool-down period including reduced exertion (e.g., stretching, meditation); and a conclusion in which the trainer offers congratulations for completing the class and explains future fitness possibilities for users. The class metadata can therefore include start times (e.g., in the form of timestamps), end times, and/or durations of these components of the class, such as including timestamps for starts of: the intro period; the warm-up period; the main exercise period; the cool down period; and the conclusion of the class. The class metadata can further include start times (or timestamps) and specifications for more specific actions occurring within these periods of the class. For example, the class metadata can further include timestamps and quantitative or qualitative representations of equipment instructions throughout the class, including: resistance settings and repetitions for a resistance training class; weight sizes and repetitions for a weightlifting class; or standing or sitting specifications, pedal speeds, and resistance settings for a spinning class.

However, a class can be annotated with any other time-referenced action, state, or state-change data stored in any other quantitative or qualitative format.

Furthermore, in one variation, a class also includes a video track, such as including: a video of a trainer exhibiting narrated exercises; graphics or animations exhibiting narrated exercises; a timer; and/or a counter (e.g., a rep counter, a step counter); etc. Thus, a computing device can both output the audio track and render the video track of this class during playback.

### 5. Initial Class Recommendations

One variation of the method S100 includes Block S101, which recites: aggregating an initial list of classes, from the corpus of prerecorded classes, that the user is predicted to complete during the first exercise session; and presenting the initial list of classes to the user through the computing device. Generally, in Block S101, the computer system can leverage user data—such as minimal self-reported fitness and demographic data of the user upon signing up on the fitness platform or historical data of classes on the fitness platform previously completed by the user—to identify a subset of classes in the class database that the user may be more likely to complete during a current or upcoming exercise session. The computer system can then present this subset of classes to the user—via the user's computing device—for selection of a class for the current or upcoming exercise session by the user.

### 5.1 New User

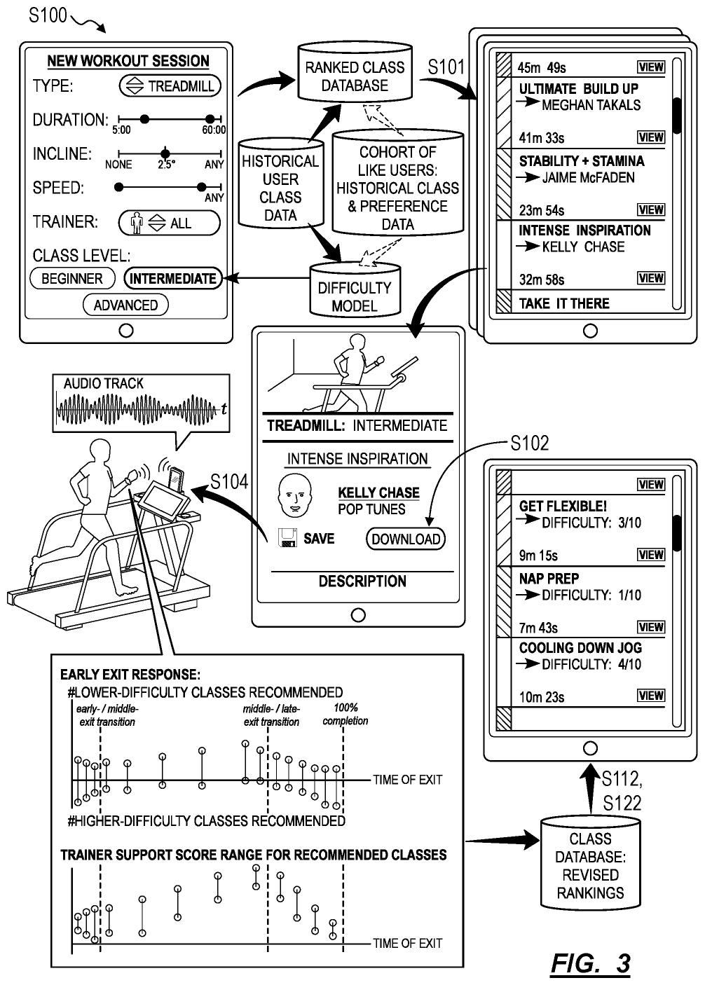

In one implementation shown in FIG. 3, the computer system leverages data collected from other users currently or previously active on the fitness platform to predict a new user's fitness-related interests and to predict classes most likely to be completed by the new user during a future exercise session.

In this implementation, when the user first installs the native application on her mobile device or navigates to a webpage associated with the fitness platform via a web browser on her mobile device, the computer system can present a survey—via the native application or webpage—to the user, such as including a prompt to indicate: the user's current fitness level (e.g., “have not exercised in years” to “just completed an IronMan”); demographic information of the user (e.g., age, occupation, gender, weight); the user's music preferences; the user's preferred exercise types (e.g., running, swimming, cycling, weight lifting, yoga); and/or a fitness-related goal. The computer system can then store these user data in a user profile associated with the user.

The computer system can then identify a cohort of other users—in a population of users on the fitness platform—exhibiting fitness levels similar to the user's manually-reported fitness level, characterized by demographics similar to the user's demographics, associated with music preferences similar to those of the user, and associated with fitness-related goals similar to those of the user. For example, the computer system can implement clustering, regression, artificial intelligence, or machine learning techniques to identify a cohort (or cluster, group) of other users who previously completed at least one class on the fitness platform and who currently exhibit (or previously exhibited) fitness levels, demographics, and/or fitness-related goals most similar to those of the user.

The computer system can then: predict a particular difficulty level that is currently appropriate for the user based on difficulty levels of classes previously completed by other users in this cohort (such as currently or at previous times when these other users exhibited greater similarity to the user); and predict a particular trainer style most appropriate for the user based on trainer styles in classes previously completed by other users in this cohort. For example, the computer system can calculate the particular difficulty level that is near difficulty levels of classes previously completed by other users in this cohort and distant from difficulty levels of classes previously started but not completed by other users in this cohort. The computer system can then rank or sort classes in the class database based on proximity of difficulty levels and associated trainer styles of these classes to the particular difficulty level and the particular trainer style thus calculated for the user.

The computer system can also filter these sorted classes to include only those that contain music in and/or near the music genre preferred by the user and of class types selected (and/or not excluded) by the user. The computer system can then: aggregate an initial list of (e.g., ten) highest-ranking classes (e.g., classes that the user is predicted to complete with greatest probability) that fulfill the user's music preferences and that are of class types preferred (or not explicitly excluded) by the user; and present this initial list of classes to the user—via the user's computing device—for selection of a first class for a first exercise session on the fitness platform.

In another example, the computer system can additionally or alternatively calculate a completion probability that the user will complete a class on the fitness platform based on classes previously completed by other users in the same cohort and repeat this process for each class on the fitness platform. The computer system can then: aggregate a subset of these classes corresponding to highest completion probabilities into an initial list of classes for the user; present this initial list of classes—ordered according to completion probabilities of these classes—to the user through the user's computing device; and filter this initial list of classes based on class types, music preferences, exercise session durations, and/or other filter parameters entered by the user.

Therefore, the computer system can: access a limited amount of information from the user via a survey when the user creates a new user profile on the fitness platform; match this user to a cohort of other similar users based on results of this survey; rank classes on the fitness platform according to probabilities that the user will complete these classes—given the user's current fitness level, etc.—based on characteristics of classes previously completed by other users in this cohort; and then serve or recommend top-ranking (e.g., ten top-ranking) classes on the fitness platform to the user in preparation for a next (e.g., first) exercise session with this user.

### 5.2 Known User

In another implementation, the computer system leverages historical data of the user active on the fitness platform to predict classes most likely to be completed by the user during the current or upcoming exercise session.

For example, the fitness platform can store metadata of classes previously started, exited (or “abandoned early”), completed, and skipped or muted by the user, such as including: difficulty levels; music genres; trainer styles; trainer support scores (e.g., a quantitative or qualitative representation of a trainer's friendliness, encouragement, and/or antagonism during narration of a class); class types; exercise types; proportions completed by the user; etc. of these classes. The computer system can then: implement regression techniques to derive a trajectory of difficulty levels of classes completed by the user; and estimate a target difficulty level of a class that is likely to be completed by the user during a next exercise session. The computer system can also: refine the user's music preferences based on genres and other stored characteristics of song titles present in classes previously completed by the user (and in classes previously abandoned by the user); estimate the user's preferences for trainer style and trainer support based on trainer demographics and trainer support scores of classes previously completed by the user (and in classes previously abandoned by the user); and estimate the user's preferences and dislike for particular exercises based on exercises in classes previously completed by the user and based on exercises in classes previously abandoned by the user, such as described below. Furthermore, the computer system can derive trends in class types completed by the user and predict a class type most likely to be selected by the user during the next exercise session.

The computer system can then rank, sort, or score classes in the class database based on: proximity of their difficulty levels to the target difficulty level calculated for the user; proximity of song titles to derived music preferences of the user; proximity of trainer styles and trainer support scores in these classes to the derived trainer preferences of the user; and presence of exercise types preferred by the user (and absence of exercise types disliked by the user). The computer system can also filter ranked classes by the user's preferred class type(s).

The computer system can then aggregate an initial list of (e.g., ten) highest-ranking classes and present this initial list of classes to the user—via the user's computing device—for selection of a class for a next exercise session on the fitness platform.

(In the foregoing implementation, the computer system can: aggregate similar data for users in a cohort; implement similar methods and techniques to calculate a target difficulty level, music preferences, trainer style and support preferences, class type preferences, and/or exercise type preferences of this cohort; and leverage these data to aggregate an initial list of classes to recommend to a new user matched to this cohort.)

However, the computer system can implement any other method or technique to generate an initial list of classes to recommend to the user in Block S110.

### 5.3 Initial Class List Generation

Furthermore, the computer system can execute the foregoing methods and techniques in real-time to aggregate this initial list of classes for the user when the user accesses the fitness platform (e.g., via the native application or via a web browser) at the beginning of a exercise session (or in preparation for an upcoming exercise session, such as on the following morning), as shown in FIG. 3.

For example, when the user accesses the fitness platform via the native application at the start of an exercise session, the computer system (or the native application) can: access a list of difficulties of classes—in the class database—completed by the user prior to the exercise session; estimate a target difficulty for the exercise session based on this list of difficulties; and query the user—via the native application—for an available exercise time for the exercise session. (The computer system or the native application can additionally or alternatively query the user to enter or confirm her preferred difficulty for this exercise session.) The computer system can then calculate a ranking of classes—in the class database—based on: proximity of difficulty levels associated with these classes to the target difficulty calculated for the user; and proximity of durations of these classes to the available exercise time specified by the user. The computer system can then interface with the native application to present classes—in the class database—to the user according to this ranking in (near) real-time in Block S101.

Alternatively, the computer system can execute the foregoing methods and techniques intermittently—such as once per day or once per week—and can queue this initial list of classes for presentation to the user when the user next accesses the fitness platform.

### 5.4 Stored Class Ranking

As described below and shown in FIG. 3, the computer system can also store rankings of classes in the class database thus calculated for the user. Responsive to early abandonment of a class by the user during an exercise session, the computer system can also: filter (e.g., by duration, by class type, difficulty level, trainer style, or trainer support score) ranked classes in the class database; and then recommend highest-ranking classes—remaining in this filtered, ranked class database—to the user for completion during the remainder of this exercise session.

### 6. Class Selection

Block S102 of the method S100 recites, at a computing device accessed by the user at a start of an exercise session, receiving selection of a first class—spanning a first duration of time, comprising a first audio track of a first trainer narrating a first exercise routine of a first type, and associated with a first difficulty level—from a corpus of prerecorded classes; and Block S104 of the method S100 recites serving the first class to the user through the computing device. Generally, in Block S102, the computer system can interface with the user through the native application (or through a web browser) executing on the user's computing device to present a variety of class options (or the initial list of classes more specifically) to the user and to receive selection of a class for the current exercise session from the user. In Block S104, the computer system can download or stream the selected class to the user's computing device.

In one implementation shown in FIGS. 1 and 3, the user's computing device presents the initial list of classes aggregated for the user by the computer system in Block S101, such as the ten highest-ranking classes selected for the user based on historical data of the user and/or other similar users on the fitness platform. If the user scrolls past this initial list of classes, the computer system can interface with the user's computing device to present next-highest ranking classes to the user. The user's computing device can also capture filter parameters entered by the user, such as: maximum difficulty level; class duration; class type; and/or music genre. The computer system and/or the user's computing device can then filter classes ranked for the user according to these filter parameters and present to the user the highest-ranking classes that meet these filter parameters.

Once the user selects a class for the current (or upcoming) exercise session, the user's computing device (e.g., the native application, a web browser) can download, stream, or otherwise access the selected class from the class database and initialize playback of the selected class for the user, as shown in FIG. 1. For example, in Block S104, the user's computing device can replay an audio track of a trainer narrating an exercise routine in the first class. (In the variation described above in which the first class contains a video track, the user's computing device can also replay a video of the first trainer exhibiting this exercise routine.) As the user's computing device replays the first class from the intro period forward in Block S104, the user may follow along to the trainer's instructions and demonstrations to perform exercises described in the first class.

### 7. Early Exit

Blocks S110 and S112 of the method S100 recite, in response to the user exiting the first class at the computing device within a first time window during replay of the first class: aggregating a first list of classes, from the corpus of prerecorded classes, including audio tracks of trainers narrating exercise routines of the first type; and presenting the first list of classes to the user through the computing device. Generally, after selecting the first class, the user may preview the first class during playback of the intro of the first class and thus make a quick decision whether to continue with the first class or elect a different class. A probability that the user will abandon (or “prematurely exit,” “opt out of”) the first class may therefore be relatively high (e.g., over 40%) at the start of the first class. However, the user may exhibit low expectations for continuing with the first class and may feel minimal investment in the first class; this early exit from the first class may therefore yield minimal frustration for the user as shown in FIG. 2, minimal risk that the user will disengage with the computer system in the short-term, and low risk that the user will lose interest in her fitness goal in the longer-term.

For example, the user may view the intro period of the first class and then quickly determine whether to continue with the first class, such as based on whether the user believes the proposed difficulty of the first class is appropriate for her current fitness level or current interest or based on whether the user likes the trainer, the music, or the exercises described. If the user thus exits the first class during this introductory period or soon after this introductory period during playback of the first class, the computer system can: predict that this early exit was triggered by the user's aversion to or lack of interest in the first class; predict that the user intends to select an alternate class; compile a short list of classes that may be (better) suited to the user's current preferences in Block Silo; and then serve this list of classes to the user in (near) real-time in Block S112, thereby rapidly assisting the user in selecting a class that the user is more likely to complete during the current exercise session.

### 7.1 Early Exit Definition

In one implementation, the computer system applies a generic duration from the start of the first class to define an early exit from the first class. For example, the computer system can access a preset class survey offset time—generic for classes in the class database—(e.g., one minute, two minutes); and then define the early exit window that extends from a start of the first class by the preset class survey offset time.

Alternatively, the computer system can define an early exit window for the first class based on metadata stored within the first class, as shown in FIG. 2. For example, the computer system can define an early exit window that: spans the beginning of the first class up to a start time of the warm-up period (i.e., the end of the intro period) in the first class, as defined in metadata; extends from the beginning of the first class up to ten seconds after the start time of the warm-up period in the first class (as shown in FIG. 2); or spans a proportion of the total duration of the first class (e.g., 2%, or one minute of a forty-minute class) from the start of the first class. For example, the computer system can: access a timeseries of events (e.g., “trainer introduction,” “class description,” “warm=up—first exercise,” “second exercise,” . . . , “cool down,” “summary and wrap up) occurring during the first class; identify a transition time—in the first class—proximal a transition from a class introduction to a first exercise (e.g., a warm-up) in the first class based on the timeseries of events; and then define the early exit window for the first class that extends from a start of the first class to this transition time.

However, the computer system can define the early exit window in any other way. The computer system can then store this early exit window in the metadata for the first class.

### 7.2 Early Exit Handling

Therefore, if the user exits the first class during this early exit window while the first class is replaying on her computing device, the computer system can aggregate a short list of (e.g., three) alternate classes that may be better suited to the user, as shown in FIGS. 1, 2, and 3.

In one implementation, the computer system interprets exit from the first class by the user within the early exit window as the user surveying the first class. Accordingly, the computer system: aggregates a first list of classes—from the class database—that includes audio tracks of trainers narrating exercise routines of the same type as the first class, of durations approximating the first duration of the first class, and labeled with difficulty levels approximating the first difficulty of the first class; and presents this first list of classes to the user responsive to interpreting exit from the first class by the user as the user surveying the first class. Alternatively, the computer system can return the initial list of classes—generated in Block S101, less the first class—to the user via a class selection menu in the native application when the user exits the first class during the early exit window.

Additionally or alternatively, in response to the user exiting the first class early, the computer system can compile a short list of alternate classes: of more, similar, and less difficulty than the first class; hosted by trainers other than the trainer in the first class; and containing a genre of music other than the music genre in the first class in Block Silo.

For example, because exit from the first class during the early exit window may correspond to the user surveying the class and minimal user investment into the first class, the computer system can interpret this early exit as lower confidence in the initial list of classes previously recommended to the user. Accordingly, the computer system can expand a range of characteristics of classes presented to the user following this early exit in order to increase probability that at least one class presented to the user better matches the user's current interests. In this example, the computer system can increase a range of difficulty levels of classes in the first list of classes, including both classes with difficulty levels less than the first difficulty level of the first class and classes with difficulty levels greater than the first difficulty level of the first class. (Conversely, responsive to an exit from the first class in the middle exit window, the computer system can predict excessive difficulty as a cause of this middle exit by the user and recommend predominantly or only classes of lower difficulty levels to the user in order to guide the user toward recovering from this middle exit.) Similarly, in this example, early exit from the first class may indicate dissatisfaction with the trainer's style or music played back in the first class. Therefore, the computing device can additionally or alternatively populate the first list of classes: with classes narrated by a wider range of trainers or by trainers with a wider range of demographic characteristics; with classes labeled with a wider range of trainer support scores; and/or with classes spanning a wider range of music genres or music characteristics (e.g., tempo).

In another implementation, the computer system stores user demographics reported manually by the user upon signup to the fitness platform and collects additional user data as the user selects and completes classes on the fitness platform over time, such as the user's preferences for exercise types, music genres, class difficulties, and trainer styles and demographics. When the user exits the first class early, the computer system can: compare the stored demographic and preference data of the user to similar data of other users on the fitness platform to identify a cohort of similar users on the fitness platform; filter this cohort of similar users to identify a subset of similar users who also exited the first class before completion and/or who exited classes of similar difficulty, trainer support scores, trainer demographics, etc. before completion; and then aggregate a small set of alternate classes that were completed by these similar users and/or that received positive reviews from these similar users. In particular, the computer system can: identify other users who, like the user, previously exhibited aversion to the first class; identify alternate classes for which these other users exhibited a preference in Block Silo; and present these alternate classes to the user in Block S112.

However, the computer system can aggregate a (short) list of alternate classes for the user in any other way or according to any other schema responsive to an early exit from the first class.

### 8. Middle Exit

Blocks S120 and S122 of the method S100 recite, in response to the user exiting the first class at the computing device within a second time window during replay of the first class: aggregating a second list of classes, from the corpus of prerecorded classes, including audio tracks of trainers narrating exercise routines of difficulty levels less than the first difficulty level and spanning durations less than the first duration of time; and serving a prompt to complete a second class, from the second list of classes, during a remainder of the exercise session to the user through the computing device in Block S122.

Generally, after selecting and screening the first class as described above, the user may elect to move forward with the first class, such as including warming up according to the trainer's instructions during the warm-up period and then engaging in a primary exercise according to the trainer's instructions during the main exercise period of the first class as the first class is played back on the user's computing device. As the user thus transitions from screening the first class to engaging with the first class and thus develops a greater feeling of investment in the first class, a probability that the user will abandon the first class may drop, such as to less than a probability of 2%. However, the user may exhibit increasing personal expectations for continuing with the first class and may sense greater investment in the first class over time. A premature exit from the first class during this period—such as due to unexpected difficulty, dislike of a song selection, or aversion to a trainer in the first class—may therefore yield increasing frustration for the user (as shown in FIG. 2), increasing risk that the user disengages with the computer system in the short-term, and increasing risk that the user loses interest in her fitness goal in the longer-term.

Following such a “middle exit” from the first class, the computer system can thus execute Blocks S120 and S122 to compile and present a list of alternate classes that the user may be more likely to complete—in whole or at least in part—during the current exercise session rather than quit the computer system (e.g., the native application) entirely, as shown in FIG. 1.

### 8.1 Middle Exit Definition

In one implementation, the computer system can leverage metadata stored within the first class to define a middle exit window for the first class. For example, the computer system can define a middle exit window that: spans the warm-up period and main exercise period of the first class (i.e., from the start time of the warm-up period to the start time of the cool-down period), as defined in metadata; extends from up to ten seconds after conclusion of the intro period to one minute before the start of the cool-down period of the first class; or spans a period from after a first proportion (e.g., 2%) of the total duration of the first class up to a second proportion (e.g., 50%) of the total duration of the first class.

However, the computer system can define the middle exit window in any other way. The computer system can then store this middle exit window in the metadata for the first class.

### 8.2 Middle Exit Handling

Generally, a predominant reason for middle exits among a population of users may be excessive difficulty of selected classes, as shown in FIG. 2. Therefore, in one implementation shown in FIG. 1, if the user exits the first class during this middle exit window, the computer system can: predict excess difficulty as a cause of this middle exit; identify a set of (e.g., three) alternate classes associated with reduced difficulty levels in Block S120; and present this set of alternate classes to the user with a prompt to complete the exercise session with a class from this set. The computer system can also calculate a length of time remaining in the first class at the time of the middle exit and select alternate classes of durations approximating this length of time, such as classes of durations within 10% of or within five minutes of (and less than) the length of time remaining in the first class at the time of the middle exit. The computer system can then present to the user (e.g., via the native application or web browser executing on the user's computing device) this short list of alternate classes, communicate to the user that she still has time to complete another class during the current exercise session, and prompt the user to select an alternate class from this list in Block S122.

In the foregoing example, the computer system can also select a first subset of classes exhibiting the same exercise as the first class, a second subset of classes exhibiting cool-down or stretching exercises, and then serve these alternate classes to the user responsive to this middle exit from the first class.

In another implementation, responsive to a middle exit from the first class, the computer system can: access a list of difficulties of classes, in the corpus of prerecorded classes, previously completed by the user; estimate a target difficulty for the user based on this list of difficulties, such as by calculating an average of these difficulties; predict excess difficulty of the first class for the user if the first difficulty of the first class exceeds the target difficulty; aggregate a second list of classes containing audio tracks of trainers narrating exercise routines of difficulty levels less than the first difficulty level in response to predicting excess difficulty of the first class for the user; and then present this second list of classes to the user.

In yet another implementation, the computer system sets a target difficulty level for the user prior to the exercise session and then recommends an initial set of classes for the user based on this target difficulty level in Block S101, as described above. However, if the user exits the first class—in this initial list of classes—during the middle exit window, the computer system can reduce the target difficulty level of the user, such as by one difficulty level or by a target proportion (e.g., 10%). The computer system can similarly refine a target trainer support score for the user, such as by similarly increasing the target trainer support score for the user (e.g., in order increase access of trainer support for the user during this sensitive middle-exit period). The computer system can then recalculate a ranking of classes—in the class database—based on: proximity of difficulty levels associated with classes, in the class database, to this revised target difficulty; proximity of durations of these classes to a remaining duration of the first class at time of exit (e.g., an “available exercise time”); proximity of trainer support scores of these classes to the target trainer support score for the user; and/or proximity of song titles in these classes to the music preferences of the user; etc. The computer system can then implement methods and techniques described above to present highest-ranking classes in the class database to the user via the user's computing device. The computer system (and/or the native application) can also prompt the user to complete a class—selected from this revised set of classes—during the remainder of this exercise session.

Alternatively, the computer system can: implement similar methods and techniques to revise a difficulty level range, a class duration range, and/or a trainer support score range for the user; filter classes previously ranked by the computer system in Block S101 to generate the initial list of classes for the user according to these difficulty level range, class duration range, and/or a trainer support score range parameters; and then present highest-ranking classes in this filtered set to the user.

Conversely, in some (e.g., a lower proportion of) instances, a user may exit classes in the middle time window due to boredom or insufficient difficulty. Therefore, the computer system can: implement the foregoing methods and techniques to aggregate a list of classes with lower difficulty levels than the first class; but also aggregate a shorter list of classes with higher difficulty levels than the first class; and then present both these lower-difficulty and higher-difficulty level classes as options for completing the remainder of the exercise session following this middle exit from the first class. For example, the computer system can: predict excess difficulty of the first class for the user in response to the user exiting the first class within the middle exit window; aggregate a first quantity of (e.g., eight) classes—associated with difficulty levels less than the first difficulty level—in response to predicting excess difficulty of the first class for the user; aggregate a second quantity of (e.g., two) classes associated with difficulty levels greater than the first difficulty level; and then present both the first set of classes and the second set of classes to the user as options for completing the reminder of the exercise session following this middle exit from the first class.

### 8.3 Middle Exit Survey

Therefore, in the foregoing implementations, the computer system (or the user's computing device) can aggregate a second list of classes more likely to be of interest to the user, better matched to the user's music and other preferences, better matched to the user's support needs, and better matched to the user's fitness level or to the user's current energy level without surveying the user for her reasons for exiting the class during the middle exit time window.

Alternately, responsive to a middle exit during playback of the first class, the computer system can immediately present a brief survey to the user in order to more directly ascertain a reason for this middle exit. For example, the computer system can serve a prompt to the user (e.g., via the native application) to select from a limited set of reasons for exiting the first class, such as including: “Didn't like the music;” “Didn't like the trainer;” “Class too hard;” or “Class too easy.” In this example, if the user thus indicated that she did not like the music, the computer system can compile a list of alternate classes that contain music of a different genre and/or excluding artists represented in the first class in Block S120. Similarly, if the user thus indicated that she did not like the trainer, the computer system can compile a list of alternate classes hosted by different trainers in Block S120. The computer system can additionally or alternatively compile a list of alternate classes characterized as more difficult or less difficult in Block S120 responsive to indication that the first class was too easy or too difficult, respectively, by the user.

### 8.4 Native Indicators

The computer system can additionally or alternatively leverage a time that the user exited the first class and timestamps of trainer actions, song changes, instructions for action or exertion changes, etc. during the first class—stored in metadata—to predict similar reasons for the user's middle exit from the first class.

For example, the computer system can predict that the user exited the first class due to an aversion to music in the first class if the time of the exit fell within ten seconds of a new song starting in the first class. More specifically, users may exit classes as a result of dislike for music contained in these classes. Accordingly, the computer system can present alternative classes—containing different song titles, artists, and/or music genres—to the user following a middle exit from the first class. In one implementation, the computer system can: access a list of start times of song titles in the first class; identify a particular song title played-back during the first class at a time of exit from the first class by the user; predict exit from the first class by the user responsive to playback of the particular song title based on proximity of a start time of the particular song title—indicated in the list of song title start times—to the time of exit from the first class; and then aggregate a second list of classes containing song titles excluding the particular song title. For example, the computer system can: estimate a correlation between the middle exit from the first class and the particular song title based on proximity of the time of exit to the start time of the particular song title (and if the time of exit succeeds the start time of the particular song title); and populate the second set of classes with classes excluding the particular song title if this correlation exceeds a threshold score.

The computer system can implement similar methods and techniques to estimate correlations between the middle exit from the first class and an artist associated with the particular song title and/or between the middle exit from the first class and a genre of the particular song title and then selectively exclude classes with song titles by the artist or in this genre from recommended class alternatives for the user.

In another example, the computer system can predict that the user exited the first class due to excessive difficulty if the time of the exit fell within ten seconds of a call for increased intensity during a main exercise period in the first class.

The computer system can then implement similar methods and techniques to select a small set of alternate classes that the user may be more likely to complete and to serve this small set of alternate classes to the user in Blocks S120 and S122.

### 8.5 Variation: Biometric Data

In one variation, in which the user wears a wearable device (e.g., a smartwatch) including a biometric sensor during the exercise session, the computer system can collect biosignal data from this wearable device and leverage this biosignal data to predict a reason for a middle exit by the user.

In one implementation in which the wearable device includes a heart rate sensor, the computer system can monitor the user's heart rate throughout the first class. Then, if the user exhibited only a small or moderate increase in heart rate from the user's resting heart rate (e.g., detected at the beginning of the exercise period) at the time of a middle exit, the computer system can: predict that the class was too easy or monotonous for the user; and then select a short list of alternate classes presenting the same type of exercise but with greater difficulty in Block S120. However, if the user exhibited a large increase in heart rate from the user's resting heart rate (or if the user's heart rate is relatively high) at the time of a middle exit, the computer system can: predict that the first class was too difficult for the user; and then compile a list of alternate classes characterized by lesser difficulty, such as one class presenting the same type of exercise at lesser difficulty, one stretching or light yoga class, and one meditation class in Block S120, thereby providing the user with various lower-intensity options for completing the current exercise session.

In another implementation in which the wearable device includes a galvanic skin response sensor, the computer system can: monitor the user's electrodermal activity via outputs of the galvanic skin response sensor; interpret magnitudes of the user's emotional state based on this electrodermal activity; and generate a timeseries of emotional magnitudes of the user over time since the start of the exercise session accordingly. The computer system can also calculate derivatives of this timeseries of emotional magnitude to generate a timeseries of rate of emotional change of the user over time since the start of the exercise session. In this implementation, the computer system can predict that a middle exit was triggered by excessive difficulty if the user exhibited a relatively consistent rate of emotional change up to the time of this middle exit; the computer system can then compile a list of lower-difficulty classes for the user in Block S120.

The computer system can also predict that a middle exit was triggered by a particular event witnessed by the user if the user exhibited a spike in rate of emotional change of the user at or near the time of the middle exit. For example, if metadata associated with the first class indicates that a particular song within the first class started just before the user exited the first class and if the computer system detected a rapid increase in emotional state of the user—based on electrodermal activity data collected by the galvanic skin response sensor—just before the user exited the first class, the computer system can correlate the user's middle exit from the first class with the particular song rather than with the difficulty of the first class or other event within the first class. When compiling a list of alternate classes for the user in Block S120, the computer system can thus select classes: of difficulty similar to that of the first class; and specifically omitting the particular song and other songs by the same artist (and songs by other related artists) in Block S120.

However, in this example, if the computer system determines from metadata associated with the first class that the user's middle exit from the first class occurred substantially outside of a transition between songs in the first class, the computer system can then: scan the metadata for timestamps of explicit lyrics in a particular song playing in the first class during the user's middle exit. If the computer system thus identifies from the metadata that an explicit lyric in the particular song is known to have occurred just before the user's middle exit from the first class, the computer system can thus select classes: of difficulty similar to that of the first class; and omitting songs containing explicit lyrics (or containing less-explicit lyrics) in Block S120.

The computer system can thus: leverage both a spike in the user's rate of emotional change and metadata relating song titles, song lyrics, and/or song characteristics to time within the first class in order to predict a correlation between characteristics of music in the first class and the user's middle exit from the first class; and then aggregate a short list of alternate classes containing music excluding such characteristics accordingly in Block S120. However, in this example, if the computer system fails to derive such a correlation between characteristics of music in the first class and the user's middle exit from the first class, the computer system can: link the spike in the user's rate of emotional change to the user's middle exit from the first class; predict a correlation between the spike in the user's rate of emotional change and the trainer (e.g., a comment, action, ore request from the trainer); and then aggregate a short list of alternate classes not hosted by the same trainer in Block S120.

However, the computer system can leverage biometric data from the user in any other way to predict a reason for the user's middle exit from the first class and to select a set of alternate classes accordingly in Block S120.

### 9. Variation: Exit Spectrum

In one variation, rather than implement rigid, non-overlapping early and middle exit windows in Blocks S110 and S120, the computer system can implement a spectrum of responses to a premature exit from the first class as a function of the time in the first class at which the exit occurred.

In one implementation, the computer system: executes both Block S110 to select a first set of alternate classes responsive to an early exit and Block S120 to select a second set of alternate classes responsive to a middle exit; and then compiles these first and second sets of alternate classes before presenting this list of alternate classes to the user. For example, the first and second sets of alternate classes can each include three classes. The computer system can thus: select a first proportion of (e.g., null, one, two, or all) alternate classes from the first set based on a ratio of the duration of the intro period viewed to the duration of the main exercise period viewed during replay of the first class; select a second proportion of (e.g., all, two, one, or null) alternate classes from the second set based on a ratio of the duration of the main exercise period viewed to the duration of the intro period viewed during replay of the first class; combine the select proportions of alternate classes from the first and second sets, accordingly; and then present this combination of alternate classes to the user.

Thus in this variation, the computer system can provide the user with class options that span a range of low-risk/low-frustration conditions and high-risk/high-frustration conditions based on when the user exited the first class, which may be correlated with user risk and user frustration.

In another example, in response to the user exiting the first class within the second time window, the computer system can store a time of exit of the user from the first class and select a first quantity of classes—associated with difficulty levels approximating the first difficulty level of the first class and spanning durations less than the duration of the first class—proportional to proximity of the time of exit to the early exit window. The computer system can also select a second quantity of classes—associated with difficulty levels less than the first difficulty level and spanning durations less than the duration of the first class—inversely proportional to proximity of the time of exit to the early exit window, as shown in FIG. 3. Furthermore, the computer system can select a third quantity of classes—associated with cool-down exercises and spanning durations less than the duration of the first class—proportional to proximity of the time of exit to the late exit window (described below). The computer system can then present the first quantity of classes, the second quantity of classes, and the third quantity of classes to the user through the computing device and prompt the user to select an alternate class—from the first, second, and third quantities of classes—to complete during the remainder of the current exercise session.

### 10. Alternate Class Selection

The user may then select an alternate class from the short list of alternate classes presented by the computer system (e.g., via the native application executing on the user's computing device) in Block S112 or Block S122. The computer system can then load the alternate class onto the user's computing device (or stream the alternate class to the user's computing device).

The computer system can replay the alternate class from the beginning of the intro period of the alternate class, thereby enabling the user to screen the alternate class, as described above. If the user exits the alternate class during the intro session of the alternate class, the computer system can re-execute Block Silo as described above to aggregate another list of alternate classes for the user—now with further difficulty, trainer, music, and/or other filters derived from the user's exit from the first class.

Similarly, if the user exits the alternate class during a warm-up period or main exercise period of the alternate class, the computer system can: further refine difficulty, trainer, music, and/or other filters—previously derived from the user's middle exit from the first class—based on the user's middle exit from the alternate class; and then re-execute Block S120 as described above to aggregate another list of alternate classes for the user based on these refined filters.

### 10.1 Variation: Alternate Class Start Point

In one variation, once the user selects an alternate class from the list compiled by the computer system in Block S120 and once the alternate class is loaded onto the computing device, the computer system can automatically skip select sections of the alternate class in order to enable the user to seamlessly transition from the first class to the alternate class.

In one implementation, if the user exited the first class during a main exercise period of the first class (i.e., rather than during the warm-up period), the computer system can automatically skip forward (or “jump”) to a beginning of the main exercise period in the alternate class—rather than playing back the alternate class from beginning—since the user may already be warmed-up and ready for the main exercise period. Alternatively, the computer system can: play back the intro period of the alternate class to enable the user to screen the alternate class, as described above; and then automatically jump to one minute prior to the beginning of the main exercise period in the alternate class to give the user opportunity to prepare for entering the main exercise period.

Similarly, if the user exited the first class during a warm-up period of the first class, the computer system can automatically skip forward (or “jump”) to a beginning of the warm-up period of the alternate class—rather than playing back the alternate class from beginning—in order to ensure that the user is warmed-before beginning the main exercise period of the alternate class.

However, the computer system can initiate an alternate class in any other way responsive to selection by the user.

### 11. Late Exit

Block S130 of the method S100 recites, in response to the user exiting the first class at the computing device within a third time window succeeding the first and second time windows during replay of the first class, serving congratulations for achieving the first exercise routine of the first class to the user through the computing device. Generally, after selecting the first class, reviewing the intro period, completing the warm-up, and completing all or most of a main exercise period of the first class, the user may be satisfied with her effort, may have reached her exercise goal for the current exercise session, and may thus prematurely exit the first class, such as without completing a cool-down or viewing a conclusion presented in the first class. A probability that the user will abandon the first class prior to 100% completion may therefore increase at later times within the first class; however, such late exit may be correlated with user satisfaction rather than user frustration (as shown in FIG. 2) and may therefore present minimal risk that the user will lose interest in her fitness goal in the longer-term. Therefore, because the user is less likely to exhibit frustration and is more likely to experience satisfaction with her effort following a late exit from the first class during a late exit window, the computer system can serve congratulations to the user for her effort (e.g., via the native application executing on her computing device) in Block S130 (and withhold a suggestion that the user complete an additional class during the current exercise session, since the latter may deflate the user's sense of accomplishment).

### 9.1 Late Exit Definition

In one implementation, the computer system applies a generic duration—such as ten minutes—from the end of the first class to define a late exit from the first class. Alternatively, the computer system can leverage metadata stored within the first class to define a late exit window for the first class. For example, the computer system can define a late exit window that: spans the second half of the first class (i.e., last 50% of total duration of the first class); spans a period of the first class succeeding the conclusion of the main exercise period; or extends from one minute prior to conclusion of the main exercise period to one minute from the end of the first class.

In another example, the computer system can define the late exit window to extend from a time proximal (e.g., preceding by one minute, concurrent with, or succeeding by one minute) completion of a primary exercise presented in the first class to a cool-down exercise presented later in the first class. Thus, if the user exits the first class during the late exit window, the computer system can estimate that the user completed most or all of the primary exercise presented during the first class and thus gained most of the benefit of the first class. Accordingly, the computer system can: interpret exit from the first class by the user within the late exit window and prior to conclusion of the first class as (sufficient) completion of the first class by the user; and then serve a congratulatory prompt to the user—through the computing device—accordingly.

However, the computer system can define the late exit window in any other way. The computer system can then store this late exit window in the metadata for the first class.

### 9.2 Late Exit Handling

In response to the user exiting the first class during the late exit window, the computer system can thus serve a notification to the user congratulating the user on her effort in Block S130, as shown in FIG. 1.

In one variation, the computer system also implements methods and techniques described above to survey the user following a late exit from the first class. For example, in response to the user exiting the first class within the late exit window, the computer system can survey the user—through the computing device—for her satisfaction with the first class, such as whether the user found the first class to be: “great;” “boring;” “too hard;” or “too easy” or by scoring the user's perceived difficulty completing the class on a scale of “1” to “10.” In this example, the computer system can then: adjust (or “scale”) a target difficulty level for the user based on the difficulty level of the first class and the user's survey response; and aggregate a third list of classes—associated with difficulty levels near this revised target difficulty level for the user—to recommend to the user for a next exercise session on a future date. The computer system can then: prompt the user to select a class—from this third list of classes—for the next exercise session; and queue the selected class accordingly for the user.

Furthermore, the computer system can input a record of the user completing the first class—despite the late exit—into the user's profile and then compile a list of classes that the user may be interested in during a next exercise session based on the user's updated profile. The computer system can then present this list of classes to the user (e.g., via the native application executing on the user's computing device) with a prompt to consider these classes during the user's next exercise session. The user may then flag or elect to store a class in this list for quick access in the future.

### 10. Full Completion/No Exit

Alternatively, the user may view the first class effectively in its entirety, such as: at least 98% of the duration of the first class or up to one minute from the conclusion of the first class. The computer system can then implement methods and techniques similar to those described above to: congratulate the user; survey the user; and/or present a list of classes that the user may be interested in completing during a future exercise session.

The computer systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a operator computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for responding to early abandonment of exercise by a user comprising:
at a computing device accessed by the user at a start of an exercise session, receiving selection of a first class from a corpus of prerecorded classes, the first class spanning a first duration of time, comprising a first audio track of a first trainer narrating a first exercise routine of a first type, and associated with a first difficulty level;
serving the first class to the user through the computing device;
in response to the user exiting the first class at the computing device within a first time window during replay of the first class:
aggregating a first list of classes, from the corpus of prerecorded classes, comprising audio tracks of trainers narrating exercise routines of the first type; and
presenting the first list of classes to the user through the computing device;
in response to the user exiting the first class at the computing device within a third time window succeeding the first time window during replay of the first class:
serving a congratulatory prompt, for completing the first exercise routine, to the user through the computing device; and
in response to the user exiting the first class at the computing device within a second time window between the first time window and the third time window during replay of the first class:
aggregating a second list of classes, from the corpus of prerecorded classes, comprising audio tracks of trainers narrating exercise routines of difficulty levels less than the first difficulty level and spanning durations less than the first duration of time; and
serving a prompt to select a second class, from the second list of classes, for completion during a remainder of the exercise session to the user through the computing device.

2. The method of claim 1:
further comprising:
accessing a manually-reported fitness level of the user and a set of demographics of the user;
identifying a cohort of users, in a population of users, exhibiting fitness levels proximal the manually-reported fitness level of the user and characterized by demographics proximal the set of demographics of the user;
aggregating an initial list of classes, from the corpus of prerecorded classes, that the user is predicted to complete during the first exercise session based on classes previously completed by users in the cohort of users; and
presenting the initial list of classes to the user through the computing device;
wherein receiving selection of the first class comprises receiving selection of the first class from the initial list of classes; and
wherein aggregating the second list of classes comprises selecting a second subset of classes, from the initial list of classes, comprising audio tracks of trainers narrating exercise routines of difficulty levels less than the first difficulty level of the first class.

3. The method of claim 2, wherein aggregating the first list of classes, from the corpus of prerecorded classes, comprises aggregating the first list of classes from the initial list of classes and excluding the first class.

4. The method of claim 2:
wherein aggregating the initial list of classes, from the corpus of prerecorded classes comprises:
for each class in the corpus of prerecorded classes, calculating a completion probability that the user will complete the class based on classes previously completed by users in the cohort of users; and
aggregating classes, in the corpus of prerecorded classes, corresponding to highest completion probabilities, into the initial list of classes;
wherein presenting the initial list of classes to the user comprises presenting the initial list of classes, ordered according to completion probabilities, to the user through the computing device; and
wherein aggregating the second list of classes comprises filtering the initial list of classes for difficulty levels less than the first difficulty level of the first class and durations less than the first duration of the first class.

5. The method of claim 1:
further comprising, in response to the user exiting the first class at the computing device within the second time window:
accessing a list of difficulties of classes, in the corpus of prerecorded classes, previously completed by the user;
estimating a target difficulty for the user based on the list of difficulties; and
predicting excess difficulty of the first class for the user in response to the first difficulty level of the first class exceeding the target difficulty for the user; and
wherein aggregating the second list of classes comprises aggregating the second list of classes comprising audio tracks of trainers narrating exercise routines of difficulty levels less than the first difficulty level in response to predicting excess difficulty of the first class for the user.

6. The method of claim 1:
further comprising defining the third time window, for the first class, extending from a third time proximal completion of a primary exercise presented in the first class to a cool-down exercise presented in the first class; and
wherein serving the congratulatory prompt to the user comprises:
interpreting exit from the first class by the user within the third time window and prior to a conclusion of the first class as completion of the first class by the user; and
serving the congratulatory prompt to the user, through the computing device, in response to interpreting completion of the first class by the user.

7. The method of claim 1:
further comprising:
accessing a timeseries of events during the first class;
identifying a transition time, in the first class, proximal a transition from a class introduction to a first exercise in the first class based on the timeseries of events; and
defining the first time window, for the first class, extending from a start of the first class to the transition time in the first class; and
wherein presenting the first list of classes to the user comprises:
interpreting exit from the first class by the user within the first time window as the user surveying the first class; and
presenting the first list of classes to the user in response to interpreting exit from the first class by the user as the user surveying the first class.

8. The method of claim 1:
further comprising
accessing a preset class survey offset time for classes in the corpus of prerecorded classes; and
defining the first time window, for the first class, extending from a start of the first class by the preset class survey offset time;
wherein aggregating the first list of classes comprises aggregating the first list of classes, from the corpus of prerecorded classes, comprising audio tracks of trainers narrating exercise routines of the first type, of durations approximating the first duration, and of difficulties approximating the first difficulty level; and
wherein presenting the first list of classes to the user comprises:
interpreting exit from the first class by the user within the first time window as the user surveying the first class; and
presenting the first list of classes to the user in response to interpreting exit from the first class by the user as the user surveying the first class.

9. The method of claim 1, further comprising, prior to the exercise session:
accessing a list of difficulties of classes, in the corpus of prerecorded classes, completed by the user prior to the exercise session;
estimating a target difficulty for the exercise session based on the list of difficulties;
receiving an available exercise time for the exercise session from the user;
calculating a ranking of classes, in the corpus of prerecorded classes, based on:
proximity of difficulty levels associated with classes, in the corpus of prerecorded classes, to the target difficulty;
proximity of durations of classes, in the corpus of prerecorded classes, to the available exercise time; and
presenting classes, in the corpus of prerecorded classes, to the user according to the ranking.

10. The method of claim 9:
further comprising predicting a music genre preference of the user based on genres of song titles in classes completed by the user prior to the exercise session; and
wherein calculating the ranking of classes, in the corpus of prerecorded classes, comprises calculating the ranking further based on proximity of song titles in classes, in the corpus of prerecorded classes, to the music genre preference of the user.

11. The method of claim 1:
wherein aggregating the second list of classes, from the corpus of prerecorded classes, comprises:
predicting excess difficulty of the first class for the user in response to the user exiting the first class within the second time window; and
aggregating a first set of classes, associated with difficulty levels less than the first difficulty level, in response to predicting excess difficulty of the first class for the user, the first set of classes of a first quantity;
further comprising aggregating a second set of classes, associated with difficulty levels greater than the first difficulty level, the second set of classes of a second quantity less than the first quantity; and
wherein serving the prompt to select the second class, from the second list of classes, comprises serving the prompt to select the second class from the second list of classes comprising the first set of classes and the second set of classes.

12. The method of claim 1:
further comprising:
in response to the user exiting the first class at the computing device within the third time window, surveying the user, through the computing device, for a perceived difficulty of the first class;
adjusting a target difficulty level for the user based on the perceived difficulty of the first class submitted by the user and the first difficulty level associated with the first class;
aggregating a third list of classes, from the corpus of prerecorded classes, comprising audio tracks of trainers narrating exercise routines associated with difficulty levels approximating the target difficulty level for the user; and
prompting the user to select a third class, from the third list of classes, for a second exercise session on a future date; and
wherein aggregating the second list of classes, from the corpus of prerecorded classes, comprises aggregating the second list of classes absent a survey result from the user for the first class.

13. The method of claim 1:
wherein aggregating the second list of classes, from the corpus of prerecorded classes, comprises:
calculating a time remainder of the first class at time of exit from the first class by the user; and
aggregating the second list of classes spanning durations approximating the time remainder; and
wherein serving the prompt to select the second class comprises prompting the user to complete a class, selected from the second list of classes, during the remainder of the exercise session.

14. The method of claim 1:
further comprising, in response to the user exiting the first class at the computing device within the second time window:
storing a time of exit of the user from the first class;
selecting a first quantity of classes, from the corpus of prerecorded classes, proportional to proximity of the time of exit to the first time window, the first quantity of classes associated with difficulty levels approximating the first difficulty level and spanning durations less than the first duration of time; and
selecting a third quantity of classes, from the corpus of prerecorded classes, proportional to proximity of the time of exit to the third time window, the third quantity of classes associated with cool-down exercises and spanning durations less than the first duration of time;
wherein aggregating the second list of classes, from the corpus of prerecorded classes, comprises selecting a second quantity of classes, from the corpus of prerecorded classes, inversely proportional to proximity of the time of exit to the first time window, the second quantity of classes associated with difficulty levels less than the first difficulty level and spanning durations less than the first duration of time; and
wherein serving the prompt to select the second class, from the second list of classes, to the user comprises:
presenting the first quantity of classes, the second quantity of classes, and the third quantity of classes to the user through the computing device; and
prompting the user to select the second class from the first quantity of classes, the second quantity of classes, and the third quantity of classes.

15. The method of claim 1, wherein aggregating the second list of classes, from the corpus of prerecorded classes, comprises:
accessing a list of start times of song titles in the first class;
identifying a particular song title played-back during the first class at a time of exit from the first class by the user;
predicting exit from the first class by the user responsive to playback of the particular song title based on proximity of a start time of the particular song title to the time of exit; and
in response to predicting exit from the first class by the user due to the particular song title, aggregating the second list of classes, from the corpus of prerecorded classes, comprising song titles excluding the particular song title.

16. The method of claim 1:
further comprising accessing a first trainer support score for the first class;
wherein aggregating the first list of classes comprises aggregating the first list of classes, from the corpus of prerecorded classes, associated with a range of trainer support scores greater than and less than the first trainer support score; and
wherein aggregating the second list of classes comprises aggregating the second list of classes, from the corpus of prerecorded classes, associated with trainer support scores greater than the first trainer support score.

17. The method of claim 1, further comprising:
confirming completion of a first warm-up period in the first class by the user based on a time of exit from the first class by the user; and
in response to receiving selection of the second class, from the second list of classes, by the user and in response to confirming completion of the first warm-up period in the first class by the user, initiating playback of the second class following a second warm-up period in the second class at the computing device.

18. The method of claim 1, wherein serving the first class to the user through the computing device comprises, at the computing device comprising a handheld mobile device:
replaying the first audio track of the first trainer narrating the first exercise routine; and
replaying a first video of the first trainer exhibiting the first exercise routine.

19. A method for supporting users during exercise sessions comprising:
at a first computing device accessed by a first user at a start of a first exercise session, receiving selection of a first class from a corpus of prerecorded classes, the first class spanning a first duration of time, comprising a first audio track of a first trainer narrating a first exercise routine of a first type, and associated with a first difficulty level;
serving the first class to the first user through the first computing device;
in response to the first user exiting the first class at the first computing device within a first time window during replay of the first class:
aggregating a first list of classes, from the corpus of prerecorded classes, comprising audio tracks of trainers narrating exercise routines of the first type; and
presenting the first list of classes to the first user through the first computing device;
at a third computing device accessed by a third user at a start of a third exercise session, receiving selection of the first class;
serving the first class to the third user through the third computing device;
in response to the third user exiting the first class at the third computing device within a third time window succeeding the first time window during replay of the first class, serving a congratulatory prompt, for completing the first exercise routine, to the third user through the third computing device;
at a second computing device accessed by a second user at a start of a second exercise session, receiving selection of the first class;
serving the first class to the second user through the second computing device;
in response to the second user exiting the first class at the second computing device within a second time window between the first time window and the third time window during replay of the first class:
aggregating a second list of classes, from the corpus of prerecorded classes, comprising audio tracks of trainers narrating exercise routines of difficulty levels less than the first difficulty level and spanning durations less than the first duration of time; and
serving a prompt to select a second class, from the second list of classes, for completion during a remainder of the second exercise session to the second user through the second computing device.

20. A method for responding to early abandonment of exercise by a user comprising:
serving a first class to the user through a computing device during an exercise session, the first class spanning a first duration of time, comprising a first audio track of a first trainer narrating a first exercise routine of a first type, and associated with a first difficulty level;
in response to the user exiting the first class within a first time window during replay of the first class:
aggregating a first list of classes, from a corpus of prerecorded classes, spanning durations proximal the first duration of time and comprising audio tracks of trainers narrating exercise routines of the first type; and
presenting the first list of classes to the user through the computing device;
in response to the user exiting the first class within a third time window succeeding the first time window during replay of the first class, serving a congratulatory prompt, for completing the first exercise routine, to the user; and
in response to the user exiting the first class within a second time window between the first time window and the third time window during replay of the first class:
aggregating a second list of classes, from the corpus of prerecorded classes, spanning durations less than the first duration of time and comprising audio tracks of trainers narrating exercise routines of difficulty levels less than the first difficulty level; and
serving a prompt to select a second class, from the second list of classes, for completion during a remainder of the exercise session to the user through the computing device.
